(19) United States
(12) Patent Application Publication
(10) Pub. No.: US 2023/0143339 A1
(43) Pub. Date: May 11, 2023

(54) COMPOSITION COMPRISING A FIBROUS MATERIAL

(71) Applicant: Weidmann Holding AG, Rapperswil (CH)

(72) Inventors: Julia MANSER, Uster (CH); Nadina PENEDER, Schlieren (CH); Shino YAMAMOTO, Zürich (CH)

(73) Assignee: Weidmann Holding AG, Rapperswil (CH)

(21) Appl. No.: 17/920,658

(22) PCT Filed: Apr. 23, 2021
(86) PCT No.: PCT/EP2021/060695

§ 371 Date: Oct. 21, 2022

(30) Foreign Application Priority Data
Apr. 23, 2020 (EP) 20171030.8

(51) Int. Cl. A61K 8/60; A61K 8/02; A61K 8/04; A23L 33/24; A23L 33/22

(52) U.S. Cl. CPC A61K 8/60; A61K 8/027; A61K 8/042; A23L 33/24; A23L 33/22; A61K 2800/48

(57) ABSTRACT

The present invention relates to solid compositions comprising a fibrous material of natural origin obtained from plants, wherein the fibrous material comprises micro-scaled and/or nano-scaled fibril agglomerates, and an alditol; a method to obtain such compositions; compositions obtained by this method, products comprising such compositions; and the use of such compositions as formulation aid in such products.

### FIELD OF THE INVENTION

The present invention relates to the field of biopolymers, in particular to micro-fibrillated cellulose in form of a solid composition comprising a fibrous material of natural origin obtained from plants, wherein the fibrous material comprises micro-scaled and/or nano-scaled fibril agglomerates, and an alditol; a method to obtain such compositions; compositions obtained by this method, products comprising such compositions; and the use of such compositions as formulation aid in such products.

### STATE OF THE ART

Products comprising fibers of natural origin are well known to the art. These products encompass construction materials, such as adhesives and cements, food products and personal care products. Generally, the fibers are employed in order to enhance the properties of the products, such as homogeneity, rheological and mechanical properties, and stability. The fibers may act as suspending agent, e.g. to stabilize emulsions and suspensions, as texturing agent or rheology modifier, as binder or mechanically reinforcing agent, as co-emulsifier and/or as sensory modifier. The sensory enhancement provided by such fibers may be particularly desirable in food and personal care compositions.

Many personal care compositions contain natural and/or synthetic: fibers due to their numerous beneficial sensory features, such as a pleasant application on skin, a minimal wet feeling, a slight cooling effect, the absence of greasiness as well as the absence of a tacky feeling. Furthermore, fibers can be used as a binding ingredient and usually have good drying properties and in many cases have an anti-irritant effect on the skin. EP 1 243 250 discloses the use of fibers as an anti-irritant ingredient in cosmetic or dermatological compositions. A broad selection is proposed, including synthetic fibers and fibers of natural origin.

In this context, cellulose micro- and/or nanofibrils have emerged as a particularly promising fiber material of natural origin obtained from plants. For example, WO 2016/166179 A1 discloses the use of cellulose fibrils in anti-wrinkle personal care compositions. The cellulose fibrils employed therein are obtained by a wet process requiring homogenization of the composition by means of a high-shear or a high-pressure homogenization means. The fibrils obtained by such a process are individually isolated and separated from each other and dispersed in a slurry. The slurry may be used as such or submitted to dewatering and/or drying step. The dewatered and/or dried fibrils may be re-dispersed in an aqueous phase in order to obtain the anti-wrinkle effect.

However, it is known that microfibrillated celluloses generally lose part of their properties upon drying, Hence, when the cellulose fibrils in the microfibrillated cellulose are re-dispersed in a liquid after they have been dried, it is generally impossible to entirely recover the properties of microfibrillated cellulose comprising an equivalent concentration of “never dried” cellulose fibrils. Generally, the rheological, mechanical, emulsifying and stabilizing properties of microfibrillated cellulose is significantly affected or even lost. This hints at irreversible changes in the structure of the fibrils and in the inter-fibril association upon drying.

U.S. Pat. No. 4,481,076 discloses re-dispersible cellulose fibrils which are obtained by the addition of a dispersion agent to microfibrillated cellulose, wherein the dispersion agent is capable of substantially inhibiting hydrogen bonding between the cellulose fibrils, and wherein the microfibrillated cellulose has been obtained by a high shear high pressure wet process. The cellulose fibrils are characterized by having a viscosity when re-dispersed in water of at least 50% of the viscosity of microfibrillated cellulose prior to drying at an equivalent concentration of cellulose fibrils. The additive includes for example ethylene glycol, propylene glycol, di-propylene glycol, glycerol, saccharides and polysaccharides.

However, U.S. Pat. No. 4,481,076 shows that, in order to recover more than 75% the viscosity, the amount of dispersion agent to be added to microfibrillated cellulose must be at least as high as the amount of cellulose fibrils, i.e. an additive to cellulose fibrils ratio of 1 or more. U.S. Pat. No. 4,481,076 remains silent about the recovery of other properties of the re-dispersed cellulose fibrils, for example recovery of the specific surface area, or the capability to stabilize emulsions.

U.S. Pat. No. 5,487,419 A discloses a process for the production of and a composition of re-dispersible mechanically disassembled cellulose and the resultant product, referred to as microdenominated cellulose (MDC). The process is characterized by drying the MDC in the presence of a dispersion agent, the dispersion agent (or additive) comprising maltodextrin (MD), carboxymethylcellulose (CMC) and lecithin (L). U.S. Pat. No. 5,487,419 A shows that the viscosity of aqueous dispersions containing re-dispersed cellulose fibrils is significantly lower than the viscosity of never dried MDC at equivalent cellulose fibrils concentrations, over a broad range of shear rates and at a (MD+CMC+L) to MDC weight ratio of 0.79. Consequently, more microdenominated cellulose (MDC) is necessary in a formulation to obtain similar properties a.o. similar viscosity.

FR 2 769 836 A1 discloses the use of dry, re-dispersible, essentially amorphous cellulose nanofibrils as texturing and reinforcing agents for cosmetic formulation. The nanofibrils are also obtained by a high shear high pressure wet process and associated with a polyhydroxylated organic compound as dispersion agent. The ratio of the polyhydroxylated organic compound to the cellulose nanofibrils is between 0.05 and 1, more preferably between 0.05 and 0.35. The polyhydroxylated compounds include polyols, such as ethylene glycol, propylene glycol and glycerin, carbohydrates and modified carbohydrates.

FR 2 769 836 A1 shows that after re-dispersion in water of 0.43 wt.-% of a mixture of cellulose nanofibrils and CMC at a CMC to cellulose nanofibrils ratio of 0.18, the dispersion has a viscosity 0.015 to 0.60 Pa·s at a shear rate of 1 s⁻¹, depending on pH and aging. However, FR 2 769 836 A1 remains silent about the viscosity of the original, never-dried cellulose nanofibers, so that it is impossible to estimate the benefit of the selected additive in terms of the retention of the cellulose nanofibrils original properties upon drying, and about the recovery of the properties of the re-dispersed cellulose nano-fibrils

EP 1 057 477 A1 discloses cosmetic emulsions obtained by using cellulose nanofibrils as obtained according to example 1 of FR 2 769 836 A1 and comprising 85% of cellulose nanofibrils and 15% of CMC. The emulsions are obtained by applying high pressure homogenization conditions. However, EP 1 057 477 A1 remains silent about the storage stability of the emulsions obtained and about the size of the emulsion droplets obtained by employing re-dispersed nanofibrils, as well as about the benefits of these re-dispersed cellulose nanofibrils compared to never dried nanofibrils.

### DESCRIPTION OF THE INVENTION

It is an object of the present invention to provide a solid composition comprising a fibrous material, and at least one dispersion agent, that provides, upon re-dispersion in a liquid, rheological, mechanical, emulsifying and stabilizing properties that are at least similar to the properties of a suspension of the never dried fibrous material at equivalent concentration of fibrous material.

In the context of the present invention, the suspension of never dried fibrous material can be obtained from the wet comminution of previously dry comminuted plant pulp, as described in more details in the present patent document, and can be called “microfibrillated cellulose”.

The content of fibrous material is called “active matter” in this context.

The applicant has now found that the recovery of the BET specific surface area of a suspension of never dried fibrous material upon drying and re-dispersion in a liquid is tightly linked with the recovery of other properties like the viscosity, the capability to stabilize an emulsion or a suspension and the capability to act as a sensory modifier. In addition, the applicant has found that, in order to retain the properties of a suspension of never dried fibrous material upon drying and re-dispersion in a liquid, the solid composition shall comprise an alditol, and fibrous material with nano- and/or micro-scaled agglomerates as well as at least 10% xylose, and the suspension of the never dried fibrous material shall be mixed with one or more alditols, and the BET specific surface area ratio of the solid composition re-dispersed in water to the aqueous suspension of the never dried fibrous material is higher than 0.75, more preferably higher than 0.9, and most preferably higher than 0.95.

Hence, in a first aspect, the present invention provides a solid composition comprising:
a. a fibrous material of natural origin obtained from plants, the fibrous material containing more than 10 wt.-% xylose, in particular more than 15 wt.-% xylose, referred to the total weight of the fibrous material, and said fibrous material comprising micro-scaled and/or nano-scaled fibril agglomerates, wherein
   i. the micro-scaled fibril agglomerates have an average length in the range of 500 nm-1000 preferably in the range of 500 nm to 600 μm and even more preferably in the range of 500 nm to 200 μm,
   ii. the nano-scale fibril agglomerates have an average length in the range of 10 nm to 500 nm and;
b. one or more alditols.

characterized in that the BET specific surface area ratio of the solid composition re-dispersed in water to an aqueous suspension of the never dried fibrous material is higher than 0.75, more preferably higher than 0.9, and most preferably higher than 0.95. As explained in the present patent document, the BET specific surface area of the solid composition re-dispersed in water corresponds to the BET specific surface area of the fibrous material comprised in the solid composition re-dispersed in water.

The fibrous material comprises micro-scaled and/or nano-scaled fibril agglomerates, which means that the individual fibers and in particular the micro fibrils of the plant pulp have been comminuted and are present at least partially or completely separated from each other, wherein the separated micro fibrils in particular form fibril agglomerates due to mutual association. The average lengths of these agglomerates are indicated in the present patent document. The micro fibrils being present within the fibril agglomerates are completely separated from the original fiber structure of the cellulose and are interconnected with each other due to mutual adherence, such that they form a common structure, in particular a network. Within the micro-scaled and/or nano-scaled fibril agglomerates, the individual micro fibrils are strongly interacting, which means that the dissociation of the fibrils constituting the agglomerates from each other would require the use of e.g. a high-pressure homogenizer, typically by passing a dispersion of a liquid, which preferably is an aqueous medium, and of the fibrous material comprising the micro-scaled and/or nano-scaled fibril agglomerates several times through said high-pressure homogenizer. This would lead to a higher time and energy consuming manufacturing process, as well as a more complex infrastructure.

Preferably, the micro- and/or nano-scaled fibril agglomerates are “substantially free of visible isolated fibrils”, meaning that essentially all possibly present fibrils in the micro- and/or nano-scaled fibril agglomerates are associated into the micro- and/or nano-scaled fibril agglomerates. In particular, the micro- and/or nano-scaled fibril agglomerates are meant to be substantially free of visible isolated fibrils, if the visible isolated fibrils represent no more than 5 percent, more preferably no more than 1 percent of the total number of the visible micro- and/or nano-scaled fibril agglomerates.

Isolated fibrils are meant to be visible, if they can easily be identified as such, when the material is observed by means of an electron microscope, such as a transmission electron microscope, or scanning electron microscope at a magnification of 10,000 and a resolution of 100 nanometers.

The nano-scaled and/or micro-scaled fibril agglomerates confer any composition comprising said fibrous material good rheological properties, smooth consistency and silky aspect which are very advantageous for example but not exclusively in cosmetic compositions and coating formulations. Furthermore, the network structure of the micro- and/or nano-scaled fibril agglomerates allows to better trap particles, droplets of any kind of liquids or bubbles of any kind of gases, which leads a good stabilization of suspensions, emulsions and foams.

The fibrous material and in particular the micro-scaled and/or nano-scaled fibril agglomerates contain more than 10 wt % xylose, more preferably more than 15 wt % xylose referred to the total weight of the fibrous material, in particular of the dry fibrous material, or referred to the total weight of the micro-scaled and/or nano-scaled fibril agglomerates, in particular of the dry micro-scaled and/or nano-scaled fibril agglomerates.

Furthermore, the xylose content of the fibrous material originates from the plants used in the production of fibrous material. That means no xylose has been added as an additive at any time. The xylose of the fibrous material of the dry composition i.e. the xylose being already part of the plant raw material presents the advantage to be better and more homogeneously dispersed in the fibrous material respectively in the micro- and/or nano-scaled fibril agglomerates. Consequently, it requires a smaller amount of xylose in the fibrous material to achieve similar effects than a fibrous material wherein the xylose has been added as an additive.

It has surprisingly been found that the amount of xylose in the fibrous material, in particular in the micro-scaled and/or nano-scaled fibril agglomerates, is particularly responsible for the improvement of sensory features in particular smooth and silky feel of cosmetic products comprising the claimed solid composition. Additionally, it has been found that a large portion of xylose in the fibrous material, in particular in the micro-scaled and/or nano-scaled fibril agglomerates, leads to a better emulsion and/or suspension stabilization of a formulation comprising the solid composition. The sensory and stabilization properties conferred by xylose are obtained when xylose is part of the fibrous material and not added to the fibrous material in order to integrate it to the nano- and/or micro-scaled fibrils agglomerates.

The amount of xylose comprised by the fibrous material is measured according to the information provided by «T. Wolfinger, Dreidimensionale Strukturanalyse and Modellierung des Kraft-Dehnungsverhaltens von Fasergefugen, TU Dresden, Fakultat Umweltwissenschaften, Dissertation submitted in November 2016».

In the context of the present invention, the BET specific surface area of the aqueous suspension of the never dried fibrous material and the BET specific surface area of the fibrous material comprised in the solid composition re-dispersed in water are measured after a solvent exchange with ethanol, acetone and hexane, followed by drying under nitrogen flow in a first step and drying in vacuum in a second step. The details of this procedure are given hereinafter.

Preparation:

For measuring the BET specific surface area, the required amount of sample (aqueous suspension of the never dried fibrous material or solid composition re-dispersed in water) with an active matter content of 3 wt.-% is weighed out in a 50 ml Falcon tube. The minimum active matter for the measurement can be calculated according to Equation 1.

$$Required minimum active matter for measuring the BET specific surface area . \\ \qquad Equation 1$$

$$minimum active matter [ g ] = \frac{10 {m}^{2}}{expected BET specific surface [ {m}^{2} / g ]}$$

The sample is then centrifuged in a centrifuge (e.g. Hettich Rotina 380 model with 6-tube 45° fixed-angle rotor) at 10,000 rpm (RCF=12745) for 4 minutes. Thereafter, the supernatant is poured off. The now empty volume is filled with 30 ml Ethanol (95%) and stirring is carried out with a glass rod. Afterwards, the sample is homogeneously dispersed using a vortex mixer for approx. 4 min and centrifuged under the same parameters. This operation is repeated 3 times.

In a next step 30 ml Acetone (≥99.5%) are added to the pellet and the mixture is stirred with a glass rod. Afterwards, the sample is homogeneously dispersed using a vortex mixer for approx. 4 min and centrifuged under the same parameters. Thereafter, the supernatant is poured off. This operation is repeated once.

At last 30 ml Hexane (≥99.5%) are added to the pellet and the mixture is dispersed using a vortex mixer for approx. 4 min. Centrifugation is once again carried out under the same conditions and the supernatant poured off. This operation is repeated once.

Afterwards, the sample is centrifuged again under the same parameters and the supernatant is poured off. The sample is stored overnight in a closed Falcon tube and then added to a glass tube, which has been previously dried and weighed in an empty state.

Thereafter, the sample is prepared with a device like The VacPrep Degasser from Micromeretics by first drying it for 30 min at 80° C. under nitrogen flow and then for 270 min at 80° C. under vacuum.

The glass tube containing the degassed sample is then placed into a suitable measurement instrument for ascertaining nitrogen sorption isotherms, such as, for example, a Micromeretics Tri Star II Plus. The nitrogen and helium used for the measurement should have a purity of 99.999%. The measurement result is exactly specified in m²/g to one decimal place. The result value specified stems from the fundamentals of BET calculation according to Brunauer, Emmett and Teller, which are known to a person skilled in the art.

In the context of the present invention, the samples of the solid composition re-dispersed in water that have been prepared for BET specific surface area measurements, as described hereinabove, are substantially free of dispersing agent, so that the BET specific surface area of only the fibrous material comprised in the solid composition re-dispersed in water is measured. Consequently, in the present document, the BET specific surface area of the solid composition re-dispersed in a liquid corresponds to the BET specific surface area of the fibrous material comprised in the solid composition re-dispersed in a liquid.

Without being bound by theory, the applicant assumes that during the solvent exchange for the sample preparation, the dispersing agents are washed out.

By “substantially free of dispersing agent” is meant that the residual amount of dispersing agent still present within the sample is less than 10 wt.-%, more particularly less than 5 wt.-% with respect to the total weight of the active matter.

Furthermore, the applicant has found that, in order to provide the desired properties in regard to the preservation of the BET specific surface of the fibrous material comprised in the solid composition re-dispersed in water, and thus in regard to the to re-dispersion of the solid composition in a liquid, rheological, mechanical, emulsifying and stabilizing properties that are at least similar to the properties of a suspension of the never dried fibrous material at equivalent concentration of active matter, the BET specific surface area of the fibrous material comprised in the re-dispersed solid composition should be larger than 125 m²/g, preferably larger than 150 m²/g, more preferably larger than 175 m²/g, still more preferably be larger than 200 m²/g, and still more preferably larger than 250, still more preferably between 250 and 350 m2/g.

In preferred embodiments, the fibrous material is obtained from pulp from the Eucalyptus tree, more preferably from the Eucalyptus urograndis or Eucalyptus globulus tree, or from the Beech tree. These wood pulps obtained by a process as described hereafter have the advantage of providing the desired micro-scaled and/or nano-scaled fibril agglomerates and having the desired BET specific surface area at the desired xylose content.

The plant pulp is obtained by pulp production processes well known from the person skilled in the art, such as sulphate, sulphite or soda processes. Preferably, the fibrous material is obtained from plant pulp that has not been treated with any chemical reaction previously, which means that the plant pulp has not been treated with any chemical modification of the cellulose previously, for example, no carboxymethylation nor TEMPO-oxidation has occurred. Although it is known that chemical treatment of the plant pulp, such as phosphorylation or oxidation, may provide a suspension of fibrous material having excellent physicochemical properties, the use of such modified material may require specific regulatory labeling that may not be desired. As a matter of fact, the applicant has found that, if the BET specific surface area of the suspension of never dried fibrous material exceeds 125 m²/g, such chemical treatments become superfluous.

The applicant has found that low molecular weight alditols (alditols are also called sugar alcohols, polyhydric alcohols, polyalcohols, or glycitols) are superior to conventional dispersion agents, such as maltodextrins and carboxymethylcellulose in preserving the rheological, mechanical, emulsifying and stabilizing properties of a suspension of never dried fibrous material after drying and re-dispersion at equivalent concentration of active matter in a liquid. The low molecular weight alditols are especially efficient due to the micro-scaled and/or nano-scaled fibril agglomerate comprised in the fibrous material. Without being bound by theory, the applicant believes that the low molecular weight alditol penetrate better into the fibrous network structure of micro-scaled and/or nano-scaled fibril agglomerates. This contributes to better re-dispersion properties as well as a better preservation of the rheological, mechanical, emulsifying and stabilizing properties of a suspension of never dried fibrous material after drying and re-dispersion at equivalent concentration of active matter in a liquid, with a smaller amount of alditol.

In a preferred embodiment, the BET specific surface area of the never dried fibrous material is larger than 125 m2/g, such BET specific surface area confers the fibrous material a particular fine network structure for the penetration of the alditol. And an even better recovery of the properties is obtained when said never dried fibrous material is mixed with one or more alditols, with a specific alditol to fibrous material weight ratio, and the fibrous material comprises nano- and/or micro-scaled agglomerates as well as at least 10% xylose, and the BET specific surface area ratio of the fibrous material comprised in the solid composition re-dispersed in water to the aqueous suspension of the never dried fibrous material is higher than 0.75, more preferably higher than 0.9, and most preferably higher than 0.95.

Accordingly, in preferred embodiments, one or more alditols that have a molecular weight less than 196 g/mol are selected from the group consisting of glycerol (MW=92.09 g/mol), erythritol (MW=122.12 g/mol), threitol (MW=122.12), pentaerythritol (MW=136.15), xylitol (MW=152.15 g/mol), ribitol (MW=152.15 g/mol), arabinitol (MW=152.15 g/mol), mannitol (MW=182.17), sorbitol (MW=182.17), allitol (MW=182.17), altritol (MW=182.17), galactitol (MW=182.17), glucitol (MW=182.17), iditol (MW=182.17), and xylitol (MW=182.17).

Preferably and for regulatory product labelling reasons, the one or more alditols are from natural origin and obtained from plants.

The solid composition may be dispersed in various liquids, including water and organic solvents, such as alcohols, for example ethanol, isopropanol, ethylene glycols and propylene glycols, ethers, ketones, esters, for example short to middle chain esters and polar vegetable oils, and water-in-oil or oil-in-water emulsions, nano-emulsions and micro-emulsions. This allows the solid composition according to the invention to be employed in a broad range of applications.

The alditol to fibrous material weight ratio is from 0.5 to 1.1, preferably from 0.7 to 1.0, for example 0.8, 0.85, 0.90, 0.95 or 0.99, in order to obtain a BET specific surface area ratio of the fibrous material comprised in the solid composition re-dispersed in water to an aqueous suspension of the never dried fibrous material that is higher than 0.75, more preferably higher than 0.9, and most preferably higher than 0.95. If this ratio is too small, the re-dispersion of the solid composition may be not effective, whereas if it is too large, part of the desired rheological, mechanical, emulsifying and stabilizing properties may be lost. Once dispersed in a liquid, more particularly in a protic liquid that may be selected from the group consisting of water, ethanol, methanol, isopropanol, glycerol, propylene glycol, dipropylene glycol, pentandiol or mixtures thereof, the re-dispersed solid composition according to the present invention is preferably a self-standing, gel-like material at room temperature, and also for example at 20° C. or 25° C. or 30° C. In the context of the present invention, the term “self-standing, gel-like material” is used to describe a material which does essentially not flow when submitted to low shear stresses. Typically, a self-standing gel-like material does not flow under the action of gravity, for example when a vessel containing it is put upside down, as shown in FIG. 1.

In preferred embodiments, the solid composition according to the present invention, once dispersed in a protic liquid at an effective concentration, has such a consistency that it forms a self-standing, gel-like material. If the liquid is water or ethanol, the effective concentration may be 1 wt.-% or more, for example 3 wt.-% or 10 wt.-%. If the concentration is too low, then the material may flow slowly under the activity of gravitation.

Increasing the shear stress, the afore mentioned liquid dispersion may start to flow, wherein its flow behavior is characterized by a viscosity. The viscosity may depend on the magnitude of the stress. Typically, the flow behavior of a liquid dispersion is monitored by varying the shear rate imposed to the liquid dispersion and measuring the shear-dependent viscosity of the liquid dispersion. The shear rate is typically applied by the means of a rotatory object which is immersed in the liquid dispersion and the viscosity is typically derived from the magnitude of the torque of the rotatory object produced by the viscous resistance of the liquid dispersion to the rotatory motion. Such measurement methods are well known to the art. For example, the viscosity can be measured with the apparatus IKA Rotavisc Hi-Vi I (Spindle VAN SP-3, 15 rpm), at 20° C. The assertion of the recovery of the viscosity implies the use of the same viscosity method within the same conditions for the solid composition re-dispersed in water and for the corresponding suspension of the never dried fibrous material.

Alternatively, the viscosity may be measured over time at a given shear rate, providing information about the stability of the suspension properties e.g. sedimentation behavior of the liquid dispersion.

In preferred embodiments, the solid composition according to the present invention, once re-dispersed in water has a viscosity that is no less than 90%, preferably equal to or higher than the viscosity of the corresponding suspension of never dried fibrous material at equivalent active matter concentration, that was used to produce the solid composition, whatever the way the viscosity is measured and whichever shear rate or shear stress is applied. More particularly, the viscosity of a liquid dispersion of the re-dispersed solid composition is no less than 95%, preferably equal to or higher than the viscosity of the corresponding suspension of the never dried fibrous material, when measured at shear rates from 0.01 to 100 s⁻¹, at equivalent active matter concentration.

The solid composition according to the present invention may also additionally comprise a polymeric additive, preferably one or more polysaccharides selected from the group consisting of saccharose, galactose, maltodextrin, alginate, guar gum, acacia gum, karaya gum, ghatti gum, agar gum, gellan gum, sclerotium gum, Ceratonia siliqua gum; pullulans, glycans, glycoaminoglycanes, carrageenans and xanthan gum. Such polymeric additives are especially useful in tailoring the rheological and mechanical properties of liquid or gel-like products comprising the solid composition according to the present invention.

Surprisingly, the applicant has found that liquid dispersions comprising a combination of the fibrous material and the one or more alditols mentioned hereinabove and one or more polysaccharides, do not become sticky upon drying, which is often the case of polysaccharides employed alone. In particular, once dispersed in water, solid compositions additionally comprising one or more polysaccharide gums, more particularly xanthan gum, confer to the aqueous dispersion the desired beneficial rheological and mechanical properties, while preventing this liquid dispersion to form a sticky film on substrates, such as skin and hair.

The solid composition according to the present invention may additionally comprise functional ingredients that enhances the functionality of the solid composition and/or provide the solid composition with one or more additional effects in diverse applications. These functional ingredients may include cosmetic ingredients, personal care ingredients, drugs, dyes, nutraceuticals, fragrances and flavors, electrically conducting (nano)materials, dielectric (nano)materials, adhesives, adhesion promoters, tackifiers, surfactants, disintegrants or dissolution retardants, fire retardants, emulsifiers, weighting agents, effervescent agents, and preservatives.

In preferred embodiments, the one or more preservatives may be selected from the group consisting of sodium benzoate, potassium sorbate, benzoic acid, ethylhexyl glycerin (3-[(2-ethylhexyl)oxy]-1,2-propanediol, phenoxyethanol, pentandiol, dihydroxyacetic acid, salicylic acid, sorbic acid, benzyl alcohol, glyceryl caprylate/caprate, ethyl lauroyl arginate HCl (ethyl N2-dodecanoyl-L-argininate hydrochloride), gluconolactone, phenethyl alcohol, sodium levulinate, glyceryl caprylate, triethyl citrate, Rosmarinus officinalis (Rosemary) leaf extract, sorbitan caprylate and ([(1R)-1-[(3R,4S)-3,4-dihydroxyoxolan-2-yl]-2-hydroxyethyl] octanoate).

The solid composition according to the invention may be in the form of extrudate, tablets, sponges, aerogels, xerogels, sheets, flakes, granules, pellets or powder.

In a second aspect, the present invention provides a method to obtain a solid composition according to the invention, the method comprising the steps of:
a) Providing a suspension of a fibrous material of natural origin obtained from plants, wherein the fibrous material comprises micro-scaled and/or nano-scaled fibril agglomerates, wherein the micro-scaled fibril agglomerates have an average length in the range of 500 nm-1000 μm, preferably in the range of 500 nm to 600 μm and even more preferably in the range of 500 nm to 200 μm, and wherein the nano-scale fibril agglomerates have an average length in the range of 10 nm to 500 nm, characterized in that the fibrous material contains more than 10 wt.-% xylose, in particular more than 15 wt.-% xylose, referred to the total weight of the fibrous material;
b) combining one or more alditols with the suspension provided in step a) in order to form a mixture;
c) before or after step d) adding to the mixture obtained in step b) respectively to the mixture obtained in step d) one or more polymer additives, one or more functional ingredients or a combination thereof; and
d) Drying the mixture by applying a drying method involving heat, electromagnetic waves and/or vacuum in order to form the solid composition.

In regard to step a), the suspension of fibrous material is preferably obtained by performing the steps of:
i. comminuting dry pulp by mechanical means;
ii. dispersing said comminuted pulp in a liquid; and
iii. further comminuting the pulp dispersed in the liquid,

wherein the dry pulp is comminuted without the substantial addition of a liquid, preferably without the addition of any liquid. This means that the pulp might contain very small amounts of liquid in the event that the air is not completely dry, but no liquid is properly added to the pulp.

Comminuting the pulp under dry conditions, further comminuting the pulp in liquid results into the formation of a suspension of fibrous material that possesses the desired rheological, mechanical, emulsifying and stabilizing properties. This is attributed to particular micro-scaled and/or nano-scaled fibril agglomerates obtained by this way.

In regard to step d), any drying methods well known to the art may be applied, such as dehydrating, convection oven drying, freeze drying, spray drying, drum drying, belt drying, fluidized bed drying, spray coating, microwave drying, infrared drying, sunlight drying and combinations therefrom. The selection of a specific drying method may depend on the desired format of the solid composition.

Spray drying is a drying that is well known to the art. Spray drying involves nebulizing a liquid, for example a solution, a suspension or an emulsion, comprising a solid material by the mean of a nebulizing unit, for example a one-fluid nozzle, a two-fluid nozzle or a rotatory atomizer, into a chamber that is ventilated with hot air. Typically, the liquid is fed continuously into the nebulizing unit, for example by the mean of a peristaltic pump. The fast evaporation of the solvent in the chamber results in the formation of a powder comprising the solid material. The speed and efficiency of spray-drying are controlled by several parameters, such as chamber geometry, air flow, air moisture, nozzle pressure, atomizer rotation speed, liquid feed rate, solid content and viscosity of the liquid feed, inlet air temperature and outlet air temperature. The skilled in the art person will easily select the parameter set-up that is the most suitable for drying a specific product. Typically, the solvent the solution to be dried, or the continuous phase of the emulsion or of dispersion to be dried is water, but other solvents may also be employed. In case a flammable solvent, spray-drying may be performed using an inert gas as drying medium.

Typically, the inlet temperature is between 110 and 225° C., more particularly between 140° C. and 190° C. If the inlet temperature is too low, the powder obtained may be sticky and adhere to the wall of the spray dryer of form agglomerates. If the inlet temperature is too high, the solid material may undergo irreversible changes, such as oxidation or chemical degradation, may transform into an undesired, less soluble state of matter, may provoke safety issues, or be less favorable in terms of applied energy to evaporation yield balance. Typically, the difference between the inlet temperature and the outlet temperature is tailored in such a way that optimal drying efficiency for minimal air and energy consumption, while preventing powder stickiness.

The resulting spray dried powder may be agglomerated in a controlled way by recirculating it in a multistage spray-dryer. In this process, the dried particles are put in contact with new, incompletely dried and sticky particles. This leads to larger powder particle size and better flowability. The formation of agglomerates is mainly influenced by the concentration of droplets in spray cloud and the water content of droplets at the time of collision. Typically, the inlet temperature in a multistage spray drier may be lower than in a single stage spray-dryer.

Drum dryers consist of one or more heated metal rolls on the outside of which a thin layer of the solid product-containing liquid is evaporated to dryness. The dried solid is scrapped off the roll by applying a sharp hard flexible blade sitting at an angle of 15 to 30° with the surface of the roll. The vaporized moisture is removed through a vapor head above the drum. A double-drum dryer consists of two rolls, rotating in opposite directions and separated by a narrow gap of about 0.05 to about 0.1 cm in the cold state, where the liquid is applied. Typically, the surface of the roll is heated at a temperature between 100 and 120° C. either by electrical mean or by pressurized steam. The contact between the dried product and the hot metal surface does not exceed 15 s, which is short enough to prevent significant decomposition even of heat-sensitive products. The product may then be reduced in smaller pieces or granulates, spheronized, or extruded in order to form various product shapes.

In a third aspect, the present invention provides a solid composition according to the invention and obtainable by the method disclosed hereinabove.

In preferred embodiments of the present invention, the residual moisture in the solid composition may be 35 wt.-% or less, more particularly 25 wt.-% or less, still more particularly 15 wt.-% or less, still more particularly 10 wt.-% or less, for example between 5 and 10 wt.-%. If the residual moisture is too low, then the solid composition may be more difficult to disperse in a liquid, whereas, if the residual moisture is too high, the solid product may be sticky. The residual moisture may easily be controlled, for example by controlling the drying temperature, the liquid feed rate and/or the residence time of the solid material in the dryer. These procedures are well-known to the skilled in the art person. The residual moisture in a solid composition may be measured by methods well-known to the art, such as thermogravimetry, Karl Fischer titration, and nuclear magnetic spin relaxation methods.

In a fourth aspect, the present invention provides a solid product comprising the solid composition according to the first and third aspect of the present invention, wherein the product is soluble or dispersible in a liquid, more particularly in water.

Solid products that are particularly concerned by this particular aspect of the present invention are products that involve the action of dispersing a solid form into a liquid, by hydration, suspension, emulsification or simple mixing. These products may comprise from 0.01 to 100 wt.-% of the solid composition, meaning the solid composition may be admixed with other ingredients to form the products or may be used as such to fulfill the function(s) of the product.

In preferred embodiments, the product comprising a solid composition according to the present invention is an instant soup, an instant beverage, a fragrance booster, a jellifying composition, a peeling composition, a dry personal care product, a cement composition, a plaster composition, a concrete composition, an abrasive composition, an adhesive composition, a woven or non-woven sizing composition, a paper sizing composition.

The dispersion and/or dissolution profile of the solid product in a liquid may also be tailored, either by controlling the size of the solid product, for example the particle size, or by admixing a disintegrating agent or an effervescent agent in order to speed up the dissolution of dispersion process or, in the contrary, dissolution retardants, in order to slow down the dissolution process. The disintegrating and effervescent agents, and dissolution retardants may be already present in the solid composition according to the invention, as mentioned hereinabove, or admixed with this solid composition to form the product. The agents that may be present in the solid composition may be identical to or different from those admixed to the solid composition, depending on the desired dispersion of dissolution profile.

Tailored dispersion and/or dissolution of the solid product may be useful for the controlled delivery of functional ingredients.

In a fifth aspect, the invention provides a liquid or gel-like product comprising the solid composition according to the first and third aspects of the present invention in dispersed form.

Liquid or gel-like products that are particularly concerned by this particular aspect of the present invention are products having specific flow behavior or texture and/or being multiphasic. Emulsions and suspensions are particularly concerned.

Once dispersed in a liquid, particularly in water, it may be expected that the particular micro-scaled and/or nano-scaled fibril agglomerates present in the fibrous material interact and associate weakly and that this weak association is responsible for the remarkable rheological, mechanical, emulsifying and stabilizing properties of the liquid dispersion obtained therefrom.

In preferred embodiments, the liquid or gel-like product is a home care product, a personal care product.

In a particular embodiment, the liquid or gel-like product is a personal care product comprising:
a) from 0.01 to 10 wt.-%, preferably from 0.1 to 5 wt.-%, of solid composition according to the present invention
b) up to 25 wt.-%, more particularly up to 10 wt.-%, of one or more polyols, including additional alditols that may be similar to or different from the alditols comprised in the solid composition according to the present invention;
c) up to 5 wt.-%, more particularly in the range of 0.005 to 3 wt.-%, of one or more preservatives;
d) one or more functional ingredient being different from the one or more polyols mentioned under b.) and different from the one or more preservatives mentioned under c); and
e) water to complete to 100 wt.-%;

wherein the weight percentage (wt.-%) refers to the total weight of the personal care product.

In the context of the present invention, the term “personal care product” comprises generic skin care and hair care products, such as soaps, cleansing compositions, shower gels, shampoos, hair conditioners, and the like. It also comprises cosmetic products, such as creams, body milks, facial masks, make-up and decorative products, and the like. It also comprises ingredients, blends and other compositions that are used to produce such cosmetic products. The term “personal care product” also comprises cosmeceutical and pharmaceutical products that are usually applied topically. In a further embodiment, the personal care product according to the present invention is used as a gel, a jelly, a cream-gel, a serum, a sorbet, a souffle or a mousse. These particular states of matter differentiate from other products like creams, ointments, and milks in that said states of matter are characterized by different textures and aspects. In particular, these states of matter are characterized in that they retain their shape and thickness over time and as long as they have not undergone any shear stresses, but may flow and spread under the action of shear stresses, for example by rubbing the personal care product on the skin or on the hair. Self-standing behavior at rest and flow behavior under shear stress is typical of reversible network formation in the system, whereas a self-standing gel without flow behavior under shear stress is typical of irreversible network formation in the system.

Personal care products comprising the solid composition according to the invention have good drying properties, which is due to the presence of the micro-scaled and/or nano-scaled fibril agglomerates present in the solid composition. These products are fast drying, and a fast absorption is taking place. Furthermore, the personal care product normally has good coating properties. Under “fast drying” is meant a drying process that is fast enough, so that the perception of a wet skin feeling disappears within about 30 seconds, more particularly within about 20 seconds after the personal care product has been applied to the skin. Under “fast absorption” is meant that the personal care product does not leave any noticeable residues on skin after about 20 seconds, more particularly after about 10 seconds, wherein the presence of residues is typically associated with an oily feeling, a fatty feeling, a tacky feeling or the sensation of material build-up on the skin. Under “coating properties” is meant the ability of the personal care product to form a two-dimensional fibrous network upon drying, said network conferring a smooth, silky aspect to the skin or to the hair and providing the user with the impression of an enhanced protective action of the personal care product against environmental nuisances.

Additionally, the personal care product comprising the solid composition according to the invention may have different textures and aspects, depending on the level of solid composition. The textures may range from self-standing gels, as mentioned hereinabove, to free-flowing formulations. It will be easily understood by the skilled person that the level of fibrous material comprising the micro-scaled and/or nano-scaled fibril agglomerates may be chosen depending on the desired texture of the personal care product. Hence, for example, it may be desirable that the personal care product is not too viscous or not too liquid, depending on the application of the personal care product. Preferably, the amount of active matter comprised by the personal care product is determined by the standard ISO 41 19, 1995.

In the context of the present invention, the term “water” includes cosmetic specialty water such as enriched water, sea or lake water, glacier water, hydrolates etc.

In preferred embodiments, the functional and active ingredients are cosmetic ingredients or personal care ingredients and may be selected from the group consisting of polyols different from alditols, low- to high-molecular weight saccharides, cosmetic grade surfactants, fully or partially neutralized alpha-hydroxy acids, fully or partially neutralized beta-hydroxy acids, fully or partially neutralized dicarboxylic acids, fully or partially neutralized hyaluronic acid, C10-C24 fatty acids and their salts and their esters, C10-C24 fatty alcohols and their esters, glycerol ethoxylates, proteins and peptides, collagen, glycolipids, phospholipids, sphingolipids, sterols and steroids, allantoin, coffein, amino acids and their derivatives, quaternary amines, alkaline bases, flavonoids and isoflavonoids, polyphenols, anthocyanins, organic dyes, pigments, vitamins and their derivatives, terpenes and their derivatives, sesquiterpenes and their derivatives, triterpenes and their derivatives, ubiquinones, waxes, oils and butters, carbohydrates and sugar alcohols, and their derivatives, mineral and vegetal particulates, bentonites, diatomea earth, kaolin, titan dioxide, plant extracts, plant juices, essential oils and/or perfumes.

In a sixth aspect, the invention provides the use of the solid composition according to the present invention to improve the stability, and the rheological and mechanical properties of liquid products, such as structured liquids, emulsions, nano-emulsions and suspensions.

In the following, examples are given how to obtain the desired solid composition according to the present invention and to show the properties of the solid composition that are particularly relevant for the application of these solid compositions. These examples must be taken as non-limiting illustrations of the above.

### DESCRIPTION OF THE FIGURES

FIG. 1 shows self-standing gels obtained from the aqueous suspension of the never dried fibrous material and from a solid composition re-dispersed in water according to the present invention at an active matter content of 3 wt.-%.

FIG. 2 shows a self-standing gel of solid composition 2.1 re-dispersed in glycerin at an active matter concentration of 3 wt.-%.

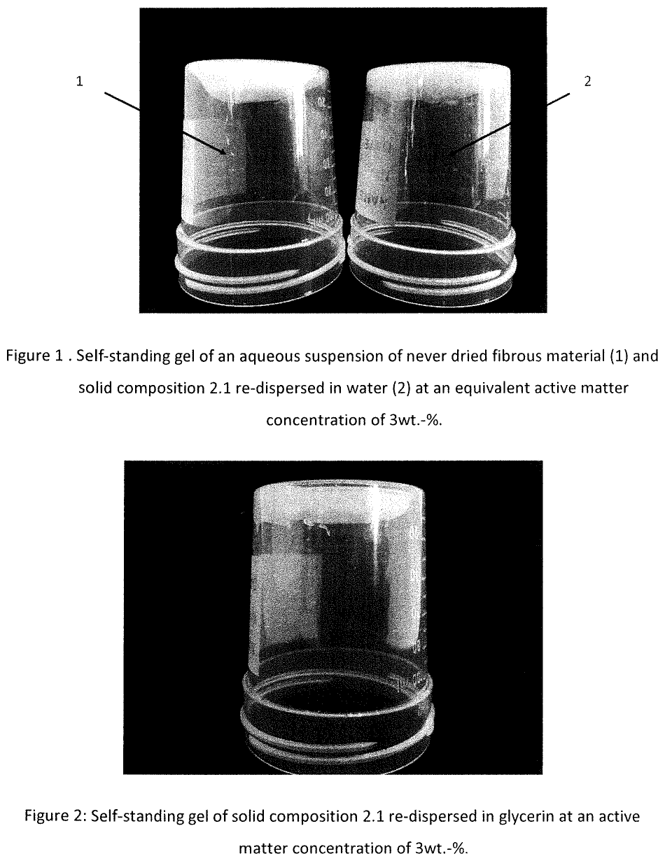

### EXAMPLES

Example 1 Preparation of solid compositions according to the present invention and comparative example

A series of solid compositions were prepared by incorporating a known amount of dispersion agent into 500 ml of an aqueous suspension of never dried fibrous material comprising 3 wt.-% active matter (Celova®, ex Weidmann Fiber Technology by Weidmann Electrical AG), under high shear mixing during 5 minutes with a rotor-stator mixer (Ultra Turrax) operating at 10,000 rpm. About 150 to 200 g of these mixtures were placed in an aluminum tray and air dried in an oven at a temperature of 90° C. until the weight of the tray reached a constant value, in order to obtain a dry cake which was reduced in the form of a powder (variant 1). Another method for drying the mixtures was to immerse the aluminum tray in liquid nitrogen followed by freeze-drying (variant 2) in order to obtain flakes.

Sample 5.1 to 6.1 (comparative example) were prepared by employing various amounts of maltodextrin as dispersion agent. Maltodextrin was previously dissolved in water at a concentration of 20 wt.-%. The compositions of these samples are given in Table 1.

Samples 1.1 to 4.1 (examples according to the present invention) were prepared by employing various amounts of glycerin 99.8% or xylitol as dispersion agent. Furthermore, a known amount of xanthan gum (Rheocare XGN, ex BASF) was added to sample 3.1 after the dispersion agent was mixed with the aqueous suspension of never dried fibrous material. The compositions after drying of these samples are given in Table 1.

For sample 7.1 an aqueous suspension of never dried fibrous material was provided with an active matter content of 3 wt.-%.

TABLE 1
Solid compositions of samples 1.1 to 6.1

| | | Solid Composition | | | | |
|---|---|---|---|---|---|---|
| | Drying | Fibrous | | | Xanthan | |
| Sample | Method | material | Glycerin | Xylitol | gum | Maltodextrin |
| 1.1 | Oven | 1 | 0.75 | | | |
| 1.2 | Freeze | 1 | 0.75 | | | |
| | Drying | | | | | |
| 2.1 | Freeze | 1 | 1 | | | |
| | Drying | | | | | |
| 3.1 | Freeze | 1 | 0.9 | | 0.1 | |
| | Drying | | | | | |
| 4.1 | Oven | 1 | | 1 | | |
| 5.1 | Freeze | 1 | | | | 1 |
| | Drying | | | | | |
| 6.1 | Oven | 1 | | | | 1.5 |

TABLE 2
Composition of sample 7.1

Composition

| Sample | Drying method | Fibrous material | Water |
|---|---|---|---|
| 7.1 | Never dried | 3 | 97 |

### Example 2 Measurement of the BET Specific Surface Area after Re-Dispersion in Water

A known amount of selected solid composition samples 1.1 to 6.1 were admixed with water with a glass stick and dispersed in water by using a rotor-stator mixer (Ultra Turrax) operating first at 9000 rpm for 3 minutes and then at 14000 rpm for 3 minutes. The sample 7.1 is an aqueous suspension of never dried fibrous material comprising 3 wt.-% of active matter so no re-dispersion in water was necessary for the BET specific surface area measurement.

The BET specific surface area of these samples was measured after having treated the samples as described hereinabove.

A parameter named “recovery” was calculated (Equation 2) from the measured data to easily compare the changes of the properties of the re-dispersed samples (1.1-6.1) to the properties of the never dried sample (7.1). The term “recovery” has been used in the application to define the change of the BET specific surface area as well as the change of viscosity in comparison to a suspension of never dried fibrous material. The term “recovery” could be also used for the comparison of other characteristic parameters of a dried and re-dispersed sample to the suspension of never dried fibrous material.

$$Calculation of recovery in function of a specific parameter P for never dried fibrous material as well as the solid composition . \qquad Equation 2$$

$$Recovery ( P ) [ \% ] = \frac{P ( solid composition )}{P ( never dried fibrous material )} \times 100 \%$$

In table 3, the recovery shows the change in the BET specific surface area of the aqueous suspension of never dried fibrous material to the re-dispersed samples 1.1-6.1. The recovery is the ratio of the BET specific surface area of the fibrous material comprised in the solid compositions re-dispersed in water to the BET specific surface area of the aqueous suspension of never dried fibrous material. The results are reported in Table 3.

TABLE 3
BET specific surface areas (SSA) of selected samples
1.1 to 6.1 after re-dispersion in water and of sample
7.1 as reference comprising the never-dried material.

| Sample | SSA [m²/g] | SSA Recovery [%] |
|---|---|---|
| 1.1 | 250 | 85.0 |
| 1.2 | 293 | 99.7 |
| 2.1 | 251 | 85.4 |
| 3.1 | 304 | 103.4 |
| 4.1 | 264 | 91.0 |
| 5.1 | 220 | 74.8 |
| 6.1 | 179 | 60.9 |
| 7.1 | 294 | — |

Table 3 shows that the recovery of the BET specific surface area is much higher for samples comprising low molecular weight alditols (1.1-4.1) in comparison to samples comprising the currently known dispersion agent maltodextrin. Even at higher maltodextrin concentrations (6.1) the desired recovery of more than 75% cannot be reached.

### Example 3 Impact of the BET Specific Surface Area of the Fibrous Material Comprised in the Solid Composition on the Stability of Emulsions

In this example, a series of emulsions have been prepared using samples 1.2, 6.1 and 7.1. The resulting emulsions are called Emulsion 1.2, Emulsion 6.1 and Emulsion 7.1 comprising a defined amount of active matter (Celova®, ex Weidmann). The composition of the formulations as well as the concentration of the active matter within the formulations are described in table 4.

In regard to the preparation of the emulsions described in table 4, following steps have been performed:

1. Preparation of water phase A
Weigh the solid compositions (samples 2.1 and sample 6.1) in deionized water in separate beakers accordingly. Weigh necessary amount of sample 7.1 in a third beaker. Beakers with water phase A are kept under stirring until the suspensions look homogeneous.

2. Addition of water phase B to water phase A
All ingredients of water phase B except deionized water are added to the beakers containing water phase A accordingly and are kept under stirring until the suspensions look homogeneous. Then deionized water was added to the beakers accordingly, again under continuous stirring and until homogeneous.

3. Heating of water phase (A+B)
The water phase A+B, prepared in step 2 is heated up to <75° C. while stirring.

4. Preparation of oil phase
Weigh the ingredients of oil phase in separate beakers. Prepare a water bath at 80° C. and place the beakers carefully and under stirring into the water bath so that no water enters the beaker until the oil phase has been melt and a clear liquid has been formed.

5. Mixing of oil phase and water phase (A+B)
The oil phase is added to the water phase (A+B) under continuous stirring to create whitish emulsions. Finally, the emulsion was homogenized at 4,000 rpm for 2 minutes.

TABLE 4
Cosmetic formulation used to test the impact of the BET specific
surface area of the fibrous material comprised in the solid
composition on the stability of known stable emulsion

| | | | Concentration in wt.-% | | |
|---|---|---|---|---|---|
| | | | Emulsion | Emulsion | Emulsion |
| Phase | Ingredient name | Supplier | 1.2 | 6.1 | 7.1 |
| Water A | Deionized water | — | 39.1 | 34.7 | |
| Water A | Solid composition sample 1.2 | Weidmann | 2.4 | | |
| Water A | Solid composition sample 6.1 | Weidmann | | 6.8 | |
| Water A | Reference sample 7.1 | Weidmann | | | 45.0 |
| Water B | Deionized water | — | 38.7 | 38.7 | 35.1 |
| Water B | Glycerine 99.8% | — | 1.8 | 1.8 | 1.8 |
| Water B | A-Leen 5 | Minasolve | 2.7 | 2.7 | 2.7 |
| Oil | Sunflower oil | — | 10.8 | 10.8 | 10.8 |
| Oil | Cutina GMS-SE | BASF | 4.5 | 4.5 | 4.5 |
| | | | 100.0 | 100.0 | 100.0 |

| | | | |
|---|---|---|---|
| Active matter in formulation [%] | 1.35 | 2.7 | 1.35 |

The quality of the emulsions obtained was assessed by optical microscopy 48 hours after the preparation of the emulsions. It is known by a person skilled in the art that oil in water emulsions are expected to show a better long-term stability the smaller and the more homogeneous the droplet size distribution is. These images are reported in Table 5 as a function of the BET specific surface areas measured in Example 2.

The microscopic images in Table 5 demonstrate that the emulsions obtained by a suspension of never dried fibrous material (7.1) as well as by the solid composition comprising glycerin (1.2) show a small and homogeneous droplet size distribution. In comparison the emulsion obtained with sample 6.1, which was prepared with double the amount of active matter due to the low recovery of SSA and viscosity, shows no homogenous droplet size distribution and coalescence of droplets has occurred. These results show that the recovery plays an essential role in the quality of an oil in water emulsion.

### Example 4 Impact of the BET Specific Surface Area of the Fibrous Material Comprised in the Solid Composition on Viscosity and Visco-Stability of Liquid Dispersions Comprising Dispersed Solid Compositions

The viscosity of selected solid composition samples was measured at 20° C. by using a viscometer IKA Rotavisc hi-vi I, equipped with a VAN-SP3 spindle operating at 1 rpm. Both the initial viscosity measured after 25 s and the variation of the viscosity over time (120 s, 300 s) were measured, in order to provide an estimate of the visco-stability of the dispersion.

The viscosity values, at t=30 s, t=120 s and t=300 s, are given in mPas in Table 6. Furthermore, the recovery of the viscosity of the re-dispersed solid composition in comparison to the suspension of never dried fibrous material (sample 7.1) is shown. As in Example 2, recovery is defined by the ratio between the viscosity of the solid composition re-dispersed in water to the viscosity of the suspension of never dried fibrous material at an equivalent active matter concentration of 2 wt.-%.

TABLE 6
Viscosity in mPas of selected samples that were re-dispersed
in water as a function of time in comparison to the
suspension of never dried fibrous material.

| | Viscosity [mPas] | | | Viscosity Recovery [%] | | |
|---|---|---|---|---|---|---|
| | after | after | after | after | after | after |
| Sample | 30 s | 120 s | 300 s | 30 s | 120 s | 300 s |
| 1.1 | 149′800 | 134′820 | 105′930 | 108.0 | 107.7 | 108.8 |
| 1.2 | 180′830 | 167′990 | 141′070 | 130.3 | 134.2 | 144.9 |
| 2.1 | 194′740 | 172′270 | 136′960 | 140.4 | 137.6 | 140.7 |
| 3.1 | 182′970 | 177′620 | 173′340 | 122.1 | 131.7 | 163.6 |
| 4.1 | 192′600 | 167′990 | 146′590 | 138.8 | 134.2 | 150.5 |
| 5.1 | 132′680 | 112′350 | 83′460 | 95.6 | 89.7 | 85.7 |
| 6.1 | 110′210 | 95′230 | 70′620 | 79.4 | 76.1 | 72.5 |
| 7.1 | 138′740 | 125′190 | 97′370 | — | — | — |

Table 6 shows that only solid compositions comprising alditols (1.1-4.1) can preserve or even improve the viscosity in comparison to the reference sample (7.1). Samples comprising maltodextrin (5.1, 6.1) show lower viscosity values compared to the suspension of never dried fibrous material (7.1) at equivalent active matter concentration.

### Example 5 Redispersion of Solid Composition in Glycerin

A known amount of solid composition 2.1 was re-dispersed in glycerin by mixing by hand. The mixture results in a self-standing gel-like material similar to the solid composition re-dispersed in water as shown in FIG. 2.

1. A solid composition comprising,
a) a fibrous material of natural origin obtained from plants, said fibrous material containing more than 10 wt.-% xylose referred to the total weight of the fibrous material and the fibrous material comprising micro-scaled and/or nano-scaled fibril agglomerates, wherein
i. the micro-scaled fibril agglomerates have an average length in the range of 500 nm-1000 μm,
ii. the nano-scale fibril agglomerates have an average length in the range of 10 nm to 500 nm; and
b) one or more alditols;
wherein the BET specific surface area ratio of the solid composition re-dispersed in water to an aqueous suspension of the never dried fibrous material is higher than 0.75.

2. The solid composition according to claim 1, wherein the BET specific surface area of the solid composition re-dispersed in a liquid is larger than 125 m²/g.

3. The solid composition according to claim 1, wherein the fibrous material is obtained from plant pulp that has not been treated with any chemical reaction.

4. The solid composition according to claim 1, wherein the fibrous material is obtained from pulp of the Eucalyptus tree or of the Beech tree.

5. The solid composition according to claim 1, wherein the one or more alditols have a molecular weight less than 196 g/mol.

6. The solid composition according to claim 1, wherein the one or more alditols are of natural origin and obtained from plants.

7. The solid composition according to claim 1, wherein the solid composition is dispersible in a liquid selected from the group consisting of water, alcohols, oils and water-in-oil or oil-in-water emulsions, nano-emulsions and micro-emulsions.

8. The solid composition according to claim 1, wherein the alditol to fibrous material weight ratio is from 0.5 to 1.1.

9. The solid composition according to claim 1, wherein said solid composition, once dispersed in a liquid, at an effective concentration, forms a self-standing, gel-like material.

10. The solid composition according to claim 1, wherein said solid composition, once re-dispersed in water has a viscosity recovery after 300 s that is no less than 95%.

11. The solid composition according to claim 1, additionally comprising one or more polymeric additive.

12. The solid composition according to claim 1, additionally comprising one or more functional ingredients selected from the group consisting of cosmetic ingredients, personal care ingredients, drugs, dyes, nutraceuticals, fragrances and flavors, electrically conducting (nano)materials, dielectric (nano)materials, adhesives, adhesion promoters, tackifiers, surfactants different from cosmetic grade surfactants, emulsifiers, weighting agents, effervescent agents, and preservatives.

13. The solid composition according to claim 12, wherein the preservative is selected from the group consisting of sodium benzoate, potassium sorbate, benzoic acid, ethylhexyl glycerin (3-[(2-ethylhexyl)oxy]-1,2-propanediol, phenoxyethanol, pentandiol, dihydroxyacetic acid, salicylic acid, sorbic acid, benzyl alcohol, glyceryl caprylate/caprate, ethyl lauroyl arginate HCl (ethyl N2-dodecanoyl-L-argininate hydrochloride), gluconolactone, phenethyl alcohol, sodium levulinate, glyceryl caprylate, triethyl citrate, Rosmarinus officinalis (Rosemary) leaf extract, sorbitan caprylate and ([(1R)-1-[(3R,4S)-3,4-dihydroxyoxolan-2-yl]-2-hydroxyethyl] octanoate).

14. The solid composition according to claim 1, wherein the solid composition is in the form of extrudate, tablets, sponges, aerogels, xerogels, sheets, flakes, granules, pellets or powder.

15. A method to obtain a solid composition, the method comprising the steps of:
a) Providing a suspension of a fibrous material of natural origin obtained from plants, wherein the fibrous material comprises micro-scaled and/or nano-scaled fibril agglomerates, wherein the micro-scaled fibril agglomerates have an average length in the range of 500 nm-1000 μm, wherein the nano-scale fibril agglomerates have an average length in the range of 10 nm to 500 nm, and wherein the fibrous material contains more than 10 wt.-% xylose referred to the total weight of the fibrous material;
b) combining one or more alditols with the suspension provided in step a) in order to form a mixture; and
c) Before or after step d), adding to the mixture obtained in step b) respectively to the mixture obtained in step d) one or more polymer additives, one or more functional ingredients or a combination thereof; and
d) Drying the mixture by applying a drying method involving heat, electromagnetic waves and/or vacuum in order to form the solid composition.

16. The method according to claim 15, wherein the suspension of fibrous material is obtained by performing the steps of:
a) comminuting dry pulp by mechanical means;
b) dispersing said comminuted pulp in a liquid; and
c) further comminuting the pulp dispersed in the liquid,
wherein the dry pulp is comminuted without the substantial addition of a liquid.

17. The method according to claim 15, wherein the drying method is selected from the group consisting of dehydrating, convection oven drying, freeze drying, spray drying, drum drying, bed drying, spray coating, microwave drying, infrared drying, sunlight drying and combinations therefrom.

18. (canceled)

19. The solid composition according to claim 1, wherein the residual moisture in the solid composition is 35 wt.-% or less.

20. A solid product comprising a solid composition that comprises
a) a fibrous material of natural origin obtained from plants, said fibrous material containing more than 10 wt.-% xylose referred to the total weight of the fibrous material and the fibrous material comprising micro-scaled and/or nano-scaled fibril agglomerates, wherein
i. the micro-scaled fibril agglomerates have an average length in the range of 500 nm-1000 μm,
ii. the nano-scale fibril agglomerates have an average length in the range of 10 nm to 500 nm; and
b) one or more alditols;
wherein the BET specific surface area ratio of the solid composition re-dispersed in water to an aqueous suspension of the never dried fibrous material is higher than 0.75,
and wherein the product is soluble or dispersible in a liquid.

21. The solid product according to claim 20, wherein the solid product is an instant soup, an instant beverage, a fragrance booster, a jellifying composition, a peeling composition, a dry personal care product, a cement composition, a plaster composition, a concrete composition, an abrasive composition, an adhesive composition, a woven or non-woven sizing composition, or a paper sizing composition.

22. A liquid or gel-like product comprising a solid composition in dispersed form, the solid composition comprising
a) a fibrous material of natural origin obtained from plants, said fibrous material containing more than 10 wt.-% xylose referred to the total weight of the fibrous material and the fibrous material comprising micro-scaled and/or nano-scaled fibril agglomerates, wherein
i. the micro-scaled fibril agglomerates have an average length in the range of 500 nm-1000 μm,
ii. the nano-scale fibril agglomerates have an average length in the range of 10 nm to 500 nm; and
b) one or more alditols
wherein the BET specific surface area ratio of the solid composition re-dispersed in water to an aqueous suspension of the never dried fibrous material is higher than 0.75.

23. The liquid or gel-like product according to claim 22, wherein the product is a home care product, or a personal care product.

24. The liquid or gel-like product according to claim 23, wherein the product is a personal care product comprising:
a) from 0.1 to 30 wt. % of the solid composition;
b) up to 25 wt. % of one or more polyols;
c) up to 5 wt. % of one or more preservatives;
d) one or more functional ingredient being different from the one or more polyols mentioned under b.) and different from the one or more preservatives mentioned under c); and
e) liquid to complete to 100 wt.-%;
wherein the weight percentage (wt.-%) refers to the total weight of the personal care product.

25. The liquid or gel-like product according to claim 24, wherein the one or more functional ingredients are selected from the group consisting of synthetic polymers, natural polymers, solvents, polyols, surfactants, fully or partially neutralized alpha-hydroxy acids, fully or partially neutralized beta-hydroxy acids, fully or partially neutralized dicarboxylic acids, fully or partially neutralized hyaluronic acid, C10-C24 fatty acids and their salts and their esters, C10-C24 fatty alcohols and their esters, glycerin ethoxylates, proteins and peptides, collagen, glycolipids, phospholipids, sphingolipids, sterols and steroids, allantoin, caffeine, amino acids and their derivatives, quaternary amines, alkaline bases, flavonoids and isoflavonoids, polyphenols, anthocyanins, organic dyes, pigments, vitamins and their derivatives, terpenes and their derivatives, sesquiterpenes and their derivatives, triterpenes and their derivatives, ubiquinones, waxes and butters, carbohydrates and sugar alcohols, and their derivatives, mineral and vegetal particulates, bentonites, diatomea earth, kaolin, titan dioxide, plant extracts, plant juices, essential oils and/or a perfumes.

26. Use of the solid composition according to claim 1 to improve the stability, and the rheological and mechanical properties of liquid products.
